(19) United States
(12) Patent Application Publication
(10) Pub. No.: US 2022/0323439 A1
(43) Pub. Date: Oct. 13, 2022

(54) URACIL DERMAL PHARMACEUTICAL FORMULATION

(71) Applicant: NANOMETICS LLC (D.B.A PHD BIOSCIENCES), La Jolla, CA (US)

(72) Inventors: Steven ISAACMAN, Oak Beach, NY (US); Andrew B. MAHON, San Francisco, CA (US)

(21) Appl. No.: 17/634,610

(22) PCT Filed: Aug. 13, 2020
(86) PCT No.: PCT/US2020/046095

§ 371 Date: Feb. 11, 2022

Related U.S. Application Data
(60) Provisional application No. 62/886,643, filed on Aug. 14, 2019.

(51) Int. Cl. A61K 31/513; A61K 9/00; A61K 47/02; A61K 47/14; A61K 47/18; A61K 47/10; A61K 47/34; A61K 47/12; A61K 47/26; A61K 47/22; A61P 17/00

(52) U.S. Cl. CPC A61K 31/513; A61K 9/0014; A61K 47/02; A61K 47/14; A61K 47/18; A61K 47/10; A61K 47/34; A61K 47/12; A61K 47/26; A61K 47/22; A61P 17/00

(57) ABSTRACT

The present disclosure provides a topical pharmaceutical formulation comprising uracil and a penetration enhancer, method of administration the same. Also provided is a method of treating or preventing dermatoses associated with the administration of a 5-fluorouracil or a precursor or prodrug thereof, such as capecitabine. the penetration enhancer is selected from the group consisting of dimethyl isosorbide, isopropyl myristate, isopropyl palmitate, octyldodecanol, oleic acid, oleyl alcohol, polyrides, pyrrolidone, thymol, tricaprylin, triolein, myristic acid, medium chain triglycerides, linoleic acid, lauric acid, glycofurol, glyceryl monooleate, ethyl oleate, dimethyl sulfoxide, dibutyl sebacate, and mixtures thereof.

### BACKGROUND

Capecitabine, originally branded as XELODA (Roche), is a widely prescribed and orally available prodrug of the chemotherapeutic agent 5-fluorouracil (5-FU). Capecitabine is indicated in the United States, Canada, and worldwide for metastatic breast cancer (mBC), adjuvant colon cancer, and metastatic colorectal cancer. It is also commonly used “off-label” to treat patients with cancers of the stomach and esophagus, amongst others.

Capecitabine is absorbed unchanged from the gastrointestinal tract and metabolized to 5-FU in three enzymatic steps. Thymidine phosphorylase (TP) is found at high concentrations in certain tumors and selectively catalyzes the third metabolic step in the activation of 5-FU to active metabolites that convey anti-cancer activity through the disruption of RNA synthesis and inhibition of thymidylate synthase.

The principle toxicity associated with capecitabine is hand-foot syndrome (HFS), also known as palmar-plantar erythrodysesthesia, and occurs in 60-70% of patients receiving capecitabine. This adverse effect limits the dose and/or length of time that capecitabine may be administered, thereby preventing the patient from receiving the optimal dose or dose schedule of capecitabine. The incidence, time to onset, and severity of HFS relates to both the dose and duration of capecitabine treatment. As treatment duration increases, the severity of HFS characteristically progresses from pain free skin changes such as erythema and edema (Grade 1 [NCI Common Terminology Criteria for Adverse Events (AEs) grading scale for HFS]) to painful changes that affect daily living (Grade 2), to severe changes such as peeling, blistering, bleeding (Grade 3), and pain requiring potent analgesics. According to the XELODA Prescribing Information (March 2015), 17-24% of patients develop Grade≥3 HFS. While the pharmacological basis for capecitabine induced HFS has yet to be fully elucidated, it is thought that the increased rates of basal cell proliferation in the palms and soles of the hands and feet, coupled with the elevated keratinocyte TP levels, are the primary causative mechanisms.

Currently, there are no approved therapies for the treatment or prophylaxis of HFS. The most effective therapeutic maneuver involves capecitabine treatment disruption or discontinuation. Typically, changes in capecitabine dosing schedule are implemented upon development of Grade 2 HFS, and there is evidence that protracted treatment interruptions or dose reduction can diminish efficacy in its approved indications.

### SUMMARY OF THE INVENTION

The invention relates to a pharmaceutical formulation comprising uracil and a penetration enhancer. This dermal formulation may be applied topically to deliver uracil to the skin, and thereby significantly delay the onset and/or progression of HFS.

In some embodiments, the topical pharmaceutical formulation comprises: about 0.05 to about 0.8% w/w Uracil, about 2.0 to about 8% w/w of a penetration enhancer, about 0.01 to about 4% w/w of an alkalizing agent, about 0.01 about 5% w/w of an antimicrobial preservative, about 10 to about 20% w/w Polyethylene Glycol, about 10 to about 20% w/w Glycerin, about 0.1 to about 3% w/w Propylene Glycol, about 0.01 to about 3% w/w of an acidifying agent, about 0.1 to about 3% w/w Carbomer, about 1 to about 5% w/w of an oily internal phase vehicle, about 1 to about 5% w/w of an ionic emulsifying agent, about 0.1 to about 7% w/w of a nonionic emulsifying agent, and about 40 to about 70% w/w water.

The invention also relates to a method of administration comprising applying about 0.08 to about 1.0 grams of the topical pharmaceutical formulation described above to a mammal. In other embodiments of the method, about 0.1 to about 0.5 grams of the topical pharmaceutical formulation is applied to a mammal.

In addition, the invention relates to a method of treating or preventing dermatoses associated with the administration of a 5-fluorouracil or a prodrug thereof in a mammal in need thereof by topically administering to the mammal in need thereof the formulation described above.

### BRIEF DESCRIPTION OF THE DRAWINGS

FIG. 1 shows the protective mechanism of a uracil formulation provided herein (UTC, which is Composition 7 in Table 3) on skill cells. FIG. 1A shows that in skin cells, 5-fluorouracil (5-FU) is converted enzymatically to toxic metabolites that cause HFS. FIG. 1B shows that Composition 7, applied to the palms and soles, results in a large local excess of concentration of uracil in the skin. Uracil is a natural substrate of the enzymes that catabolize 5-FU and prevents the formation of local cutaneous toxicity, allowing toxic species to diffuse from skin cells and reduce the likelihood of HFS.

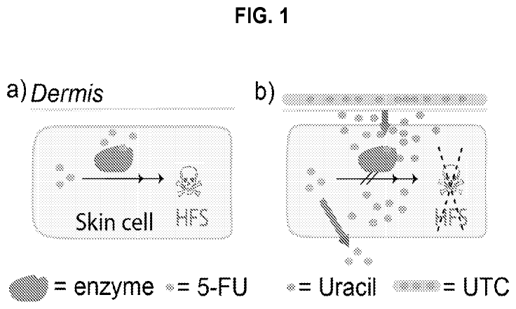

FIG. 2 shows the highest grade of HFS observed for each patient in the treatment (uracil cream-Composition 2) and placebo (PTC, which is Composition 1 in Table 1) groups in Example 2.

FIG. 3 shows a Kaplan-Meier plot of the proportion of patients without Grade≥2 HFS from randomization in each study arm of Example 2.

FIG. 4 shows human epidermal keratinocyte (HPEK) viability cultured in the presence of 5-FU and uracil. The presence of uracil protected against 5-FU induced cell death in cultured HPEK cells.

FIG. 5 shows a plot of the results of Franz diffusion experiments for uracil topical formulations with increasing concentrations of dimethyl isosorbide (DMI). The formula containing 0.3% uracil and 5% DMI outperformed all other formulations tested, even compared to the formulations that contained 15% DMI. The total amount of permeated uracil recovered from the receiver compartment at 12 h was nearly identical when comparing all formulas. The uracil formula with penetration enhancer (UTC, which is Composition 7 in Table 3) outperformed the uracil formula without penetration enhancer (1UO, which is Composition 2 in Table 1), with a 6.5-fold increase of delivered uracil when compared to the 1UO formula.

### DETAILED DESCRIPTION OF THE INVENTION

The present disclosure relates to a pharmaceutical formulation comprising uracil and a penetration enhancer. This dermal formulation may be applied topically to deliver uracil to the skin, and thereby significantly delay the onset and/or progression of HFS, a painful redness and cracking of the skin of the hands and feet, which can occur with systemic treatment with 5-fluorouracil or a precursor or prodrug thereof, such as capecitabine, or other chemotherapy agents.

Uracil is a naturally occurring metabolite and competitive substrate to enzymes that catabolize capecitabine to the toxic metabolites responsible for hand-foot syndrome (HFS). It has been demonstrated that the severity of HFS and response rates to capecitabine treatment are positively correlated, suggesting that greater drug exposure can lead to improved therapeutic outcomes; see, e.g., Chua et al., Proceedings of ASCO 22 (2003); Chua et al., Jpn J Clin Oncol 38: 244-249 (2008); Kurt et al., Acta Oncol 45: 625-626 (2006); Yun et al., J Korean Soc Coloproctol 26: 287-292 (2010); Zielinski et al., British Journal of Cancer 114: 163-170 (2016); and Clark et al., Support Cancer Ther 1: 213-218 (2004).

The pharmaceutical formulation of the present disclosure advantageously attenuates development of HFS, has enhanced aesthetic properties, and delivers an increased amount of uracil (e.g., 6.5 times more than other formulations) into the skin, i.e., uracil's site of action, without increasing the amount of uracil that permeates through the skin into systemic circulation.

Thus, administration of the formulation may allow uncompromised doses of capecitabine therapy (or other chemotherapeutic agents) to be administered for longer periods and thus enhance the therapeutic response to capecitabine treatment, without disrupting the chemotherapeutic effects of capecitabine. The uracil formulation can thereby improve patient quality of life, response rate, progression-free survival, and overall survival.

A further advantage of the present formulation is the ability for its production on a large scale (e.g., batch sizes of 75 kg) with reproducible product specification, analytical profile, and stability.

In some embodiments, the present disclosure provides a topical pharmaceutical formulation comprising: about 0.05 to about 0.8% w/w Uracil, about 2 to about 8% w/w of a penetration enhancer, about 0.01 to about 4% w/w of an alkalizing agent, about 0 to about 5% w/w of an antimicrobial preservative, about 10 to about 40% w/w of a solvent selected from the group consisting of polyethylene glycol 400, glycerin, propylene glycol, and mixtures thereof; about 0.01 to about 3% w/w of an acidifying agent, about 0.1 to about 3% w/w of a gel forming agent selected from the group consisting of a carbomer, polycarbophil, polyvinyl alcohol, povidone, hypromellose, sodium hyaluronate, hyaluronic acid, xanthan gum, pectin, methylcellulose, hydroxypropyl cellulose, hydroxyethylmethylcellulose, hydroxyethylcellulose, guar gum, dextrin, copovidone, ceratonia, carrageenan, alginic acid, carboxymethylcellulose sodium, carboxymethylcellulose calcium, ammonium alginate, sodium alginate, acacia, and potassium alginate, and mixtures thereof, about 1 to about 5% w/w of an oily internal phase vehicle, about 0 to about 5% w/w of an ionic emulsifying agent, about 0 to about 7% w/w of a nonionic emulsifying agent, and about 40 to about 70% w/w water.

In other embodiments, the present disclosure provides a topical pharmaceutical formulation comprising: about 0.05 to about 0.5% w/w Uracil, about 3 to about 6% w/w of a penetration enhancer, about 0.01 to about 4% w/w of an alkalizing agent, about 0.01 to about 5% w/w of an antimicrobial preservative, about 10 to about 20% w/w Polyethylene Glycol, about 10 to about 20% w/w Glycerin, about 0.1 to about 3% w/w Propylene Glycol, about 0.1 to about 3% w/w of an acidifying agent, about 0.1 to about 3% w/w Carbomer, about 1 to about 5% w/w of an oily internal phase vehicle, about 1 to about 5% w/w of an ionic emulsifying agent, about 0.1 to about 7% w/w of a nonionic emulsifying agent, and about 40 to about 70% w/w water.

In yet other embodiments, the present disclosure provides a topical pharmaceutical formulation comprising: about 0.05 to about 0.6% w/w Uracil, about 3.0 to about 10% w/w Dimethyl isosorbide, about 0.1 to about 2% w/w Ammonia Solution (about 29%), about 0 to about 2% w/w Methylparaben, about 0 to about 2% w/w Propylparaben, about 10 to about 20% w/w Polyethylene Glycol, about 10 to about 20% w/w Glycerin, about 0 to about 3% w/w Propylene Glycol, about 0 to about 3% w/w Hydrochloric Acid (about 20%), about 0.1 to about 5% w/w Carbomer, about 0.1 to about 2% w/w Trolamine, about 1 to about 5% w/w Dimethicone, about 1 to about 5% w/w Stearic Acid, about 0 to about 4% w/w Polysorbate, about 0 to about 3% w/w Sorbitan Monooleate, and about 40 to about 80% w/w Water.

In some embodiments, the present disclosure provides a topical pharmaceutical formulation comprising: about 0.05 to about 0.6% w/w Uracil, about 3.0 to about 10% w/w Dimethyl isosorbide, about 0.1 to about 2% w/w Ammonia Solution (about 29%); about 0 to about 2% w/w Methylparaben; about 0 to about 2% w/w Propylparaben; about 10 to about 20% w/w Polyethylene Glycol, about 10 to about 20% w/w Glycerin, about 0 to about 3% w/w Propylene Glycol, about 0 to about 3% w/w Hydrochloric Acid (about 20%), about 0.1 to about 5% w/w Carbomer, about 0.1 to about 2% w/w Trolamine, about 1 to about 5% w/w Dimethicone, about 1 to about 7% w/w a nonionic emulsifying agent, about 0 to about 5% w/w an ionic emulsifying agent, and about 40 to about 80% w/w water.

In other embodiments, the present disclosure provides a topical pharmaceutical formulation comprising: about 0.05 to about 0.6% w/w Uracil, about 3.0 to about 10% w/w Dimethyl isosorbide, about 0.1 to about 4% w/w an alkalizing agent, about 0.1 to about 2% w/w Methylparaben, about 0.01 to about 1% w/w Propylparaben, about 10 to about 40% w/w of a solvent selected from the group consisting of Polyethylene Glycol 400, Glycerin, Propylene Glycol, and mixtures thereof; about 0.01 to about 3% w/w an acidifying agent, about 0.1 to about 3% w/w Carbomer 940, about 1 to about 5% w/w Dimethicone, about 1 to about 5% w/w Stearic Acid, about 0.5 to about 4% w/w Polysorbate 80, about 0.1 to about 3% w/w Sorbitan Monooleate, and about 40 to about 60% w/w Water.

In other embodiments, the present disclosure provides a topical pharmaceutical formulation comprising: about 0.05 to about 0.5% w/w Uracil, about 3.0 to about 8% w/w Dimethyl isosorbide, about 0.1 to about 2% w/w Ammonia Solution (about 29%), about 0.1 to about 2% w/w Methylparaben, about 0.01 to about 1% w/w Propylparaben, about 10 to about 20% w/w Polyethylene Glycol 400, about 10 to about 20% w/w Glycerin, about 0.1 to about 3% w/w Propylene Glycol, about 0.01 to about 3% w/w Hydrochloric Acid (about 20%), about 0.1 to about 3% w/w Carbomer 940, about 0.1 to about 2% w/w Trolamine, about 1 to about 5% w/w Dimethicone, about 1 to about 5% w/w Stearic Acid, about 0.5 to about 4% w/w Polysorbate 80, about 0.1 to about 3% w/w Sorbitan Monooleate, and about 40 to about 60% w/w Water.

In other embodiments, the present disclosure provides a topical pharmaceutical formulation comprising: about 0.3% w/w Uracil, about 5.0% w/w Dimethyl isosorbide, about 0.9 to about 1.1 w/w Ammonia Solution (about 29%), about 0.4 to about 0.6% w/w Methylparaben, about 0.04 to about 0.06% w/w Propylparaben, about 14 to about 16% w/w Polyethylene Glycol 400, about 13 to about 15% w/w Glycerin, about 1 to about 2% w/w Propylene Glycol, about 0.01 to about 0.1% w/w Hydrochloric Acid (about 20%), about 1 to about 2% w/w Carbomer 940, about 0.4 to about 0.6% w/w Trolamine, about 3 to about 4% w/w Dimethicone, about 2 to about 3% w/w Stearic Acid, about 1 to about 2% w/w Polysorbate 80, about 0.9 to about 2% w/w Sorbitan Monooleate, and about 50 to about 60% w/w Water.

In some embodiments the formulation comprises about 0.07 to about 0.4% w/w, about 0.08 to about 0.4%, about 0.09 to about 0.38%, about 0.09 to about 0.35%, about 0.1 to about 0.3%, or about 0.1 to about 0.6% Uracil.

In some embodiments the formulation comprises about 10 to about 60% w/w, about 15 to about 60%, or about 20 to about 50% of a solvent.

In some embodiments the formulation comprises about 1.0 to about 20%, about 2 to about 15%, about 3 to about 10%, about 3.5 to about 6%, or about 4 to about 5.5%, or about 4% or about 5% w/w of a penetration enhancer.

In some embodiments the formulation comprises about 0.1 to about 4%, about 0.5 to about 3%, or about 0.9 to about 2% w/w of an alkalizing agent.

In some embodiments the formulation comprises about 0.01 to about 5%, about 0.1 to about 2%, about 0.2 to about 1.5%, about 0.4 to about 1%, or about 0.4 to about 0.8% w/w of an antimicrobial preservative.

In some embodiments the formulation comprises about 0.01 to about 5% about 0.01 to about 5%, or about 0.05 to about 4% w/w of an acidifying agent.

In some embodiments the formulation comprises about 0.1 to about 5%, about 0.9 to about 4%, or about 1 to about 2% w/w of a gel-forming agent.

In some embodiments the formulation comprises about 1 to about 10%, about 1 to about 3%, or about 3 to about 4% w/w of an oily internal phase vehicle.

In some embodiments the formulation comprises about 0.5 to about 10%, about 1 to about 5%, about 1 to about 4%, or about 2 to about 4% w/w of an ionic emulsifying agent.

In some embodiments the formulation comprises about 0.1 to about 10%, about 0.5 to about 5%, or about 0.9 to about 2% w/w of a non-ionic emulsifying agent.

In some embodiments the formulation comprises about 0.5 to about 20%, about 1 to about 15%, about 1 to about 10%, about 3.0 to about 6%, about 3.5 to about 5.5%, or about 4 to about 5.5% w/w Dimethyl isosorbide; about 0.1 to about 4%, about 0.5 to about 3%, or about 0.9 to about 1.1 w/w Ammonia Solution (about 29%); about 0.1 to about 2% w/w, about 0.3 to about 1%, or about 0.4 to about 0.6% w/w Methylparaben; about 0.01 to about 2% w/w, about 0.03 to about 1%, or about 0.04 to about 0.06% w/w Propylparaben; about 5 to about 20%, about 10 to about 18%, or about 12 to about 16% w/w Polyethylene Glycol 400; about 5 to about 20%, about 10 to about 18%, or about 12 to about 15% w/w Glycerin; about 0.1 to about 5%, about 0.5 to about 3%, or about 1 to 2% w/w Propylene Glycol; about 0.02 to about 3%, about 0.03 to about 2%, about 0.03 to about 1%, or about 0.04 to about 0.06% w/w Hydrochloric Acid (about 20%); about 0.1 to about 5%, about 0.9 to about 4%, or about 1 to about 2% w/w Carbomer 940; about 1 to about 5%, about 2 to about 4%, or about 3 to about 4% w/w Dimethicone; about 0.1 to about 2%, about 0.3 to about 1%, or about 0.4 to about 0.8% w/w Trolamine; about 1 to about 5%, about 1 to about 4%, or about 2 to about 4% w/w Stearic Acid; about 1 to about 5%, about 1 to about 4%, about 1 to about 3% w/w, or about 1 to about 2% w/w Polysorbate 80; about 0.1 to about 7%, about 0.5 to about 5%, or about 0.9 to about 2% w/w Sorbitan Monooleate; and about 40 to about 70%, about 45 to about 65%, about 50 to about 60%, or about 50 to about 56% w/w Water.

In some embodiments, the penetration enhancer is selected from the group consisting of dimethyl isosorbide (Gransolve DMI), isopropyl myristate, isopropyl palmitate, octyldodecanol, oleic acid, oleyl alcohol, polyoxylglycerides, pyrrolidone, thymol, tricaprylin, triolein, myristic acid, medium chain triglycerides, linoleic acid, lauric acid, glycofurol, glyceryl monooleate, ethyl oleate, dimethyl sulfoxide, dibutyl sebacate, and mixtures thereof.

In some embodiments, the alkalizing agent is selected from the group consisting of ammonia solution, trolamine, tromethamine, sodium hydroxide, potassium hydroxide, diethanolamine, monoethanolamine, potassium citrate, sodium citrate, sodium bicarbonate, sodium borate, sodium carbonate, potassium bicarbonate, potassium carbonate, sodium acetate, sodium phosphate, meglumine, and mixtures thereof.

In some embodiments, the antimicrobial preservative is selected from the group consisting of methylparaben, ethylparaben, propylparaben, butylparaben, benzalkonium chloride, benzethonium chloride, cetylpyridinium chloride, benzoic acid, potassium benzoate, sodium benzoate, propionic acid, sodium propionate, potassium propionate, phenoxyethanol, phenylethyl alcohol, sorbic acid, sodium lactate, lactic acid, thymol, xylitol, imidurea, hexetidine, EDTA, cresol, chloroxylenol, chlorocresol, chlorobutanol, chlorhexidine, cetrimide, calcium lactate, calcium acetate, butylene glycol, bronopol, boric acid, benzyl alcohol, and mixtures thereof.

In some embodiments, the acidifying agent is selected from the group consisting of hydrochloric acid, sulfuric acid, acetic acid, nitric acid, citric acid, propionic acid, adipic acid, lactic acid, phosphoric acid, tartaric acid, maleic acid, fumaric acid, calcium chloride, ammonium chloride, and mixtures thereof.

In some embodiments, the oily internal phase vehicle is selected from the group consisting of dimethicone, various grades of vegetable oil, mineral oil, isopropyl palmitate, octyldodecanol, oleyl alcohol, petrolatum, simethicone, tricaprylin, triolein, myristyl alcohol, medium chain triglycerides, glyceryl monooleate, ethyl oleate, dibutyl sebacate, cyclomethicone, and mixtures thereof.

In some embodiments, the ionic emulsifying agent is selected from the group consisting of stearic acid, oleic acid, palmitic acid, sodium lauryl sulfate, anionic emulsifying wax, myristic acid, linoleic acid, lecithin, lauric acid, docusate sodium, aluminum monostearate, and mixtures thereof.

In some embodiments, the nonionic emulsifying agent is selected from the group consisting of sorbitan monooleate; polyoxyethylene alkyl ethers; a polysorbate; a polyoxyethylene castor oil derivative; polyoxyethylene stearate; a polyoxylglyceride; a laurate, palmitate, stearate, trioleate, sesquioleate, dioleate, sesquiisostearate, sesquistearate, triisostearate, tri stearate, diisostearate, or monoisostearate sorbitan ester; a nonionic emulsifying wax; myristyl alcohol; a medium chain triglyceride; macrogol 15 hydroxystearate; glyceryl monooleate; cholesterol; cetyl alcohol; cetostearyl alcohol; a monoglyceride; a diglyceride; triton X-100; and mixtures thereof.

In some embodiments, the penetration enhancer is dimethyl isosorbide, and the ratio of the w/w concentrations of the uracil to dimethyl isosorbide is about 0.3 to 5. In some embodiments the concentration of uracil is about 0.3% w/w, and the concentration of dimethyl isosorbide is about 5.0% w/w.

In some embodiments, the permeation of uracil from the formulation is less than about 150.6 ng/cm², as measured using IVTP. In some embodiments, the permeation of uracil from the formulation is less than about 160.0, about 145.0 about 140.0, or about 135.0 ng/cm², as measured using IVTP.

In some embodiments the topical pharmaceutical formulation does not comprise a methyl methacrylate polymer.

In other embodiments, the formulation is an emulsion, and the viscosity of the formulation is about 100,000 to about 400,000 cps, or about 250,000 to about 320,000 cps. Viscosity may be measured with, e.g., a Brookfield LVDV II+ viscometer (Brookfield Engineerng Labs, Inc.) with a T-F spindle measured at 2 rpm for 1 min with helipath on.

As used herein an active ingredient is the component of the formulation that provides the desired pharmacological effect at the intended site of action. An active ingredient that may be used in the formulations is uracil.

As used herein, a solvent is a component of the formulation that dissolves, or helps to dissolve, one or more other components of the formulation. One solvent that may be used in the formulations is water. In addition to water, the formulation may contain one or more of the following solvents, propylene Glycol, Glycerin, various grades of polyethylene glycol (e.g., 200, 300, 400, 540, 600, 900, 1000, 1450, 1540, 2000, 3000, 3350, 4000, 4600, 8000), polyethylene oxide, poloxamer, propylene carbonate, pyrrolidone, sorbitol, xylitol, glycofurol.

As used herein, a penetration enhancer is a component of the formulation that interferes with the normal barrier properties of the skin to increase the rate at which the active ingredient is able to penetrate the skin. Penetration is the amount of drug that is delivered to and retained in the skin (i.e. the site of action) at a particular timepoint following topical application of the drug. Permeation is the amount of drug that passes through the skin into systemic circulation (systemic increases in uracil levels are not desirable so that they do not interfere with chemotherapy) and in the context of in vitro permeation test (IVPT) studies into the receiver fluid, as described in Example 5. Preferably, permeation of uracil from the topical formulations of the invention is less than 150.6 ng/cm² over a 12 h period.

The formulation preferably contains one or more penetration enhancers such as dimethyl isosorbide (Gransolve DMI), isopropyl myristate, isopropyl palmitate, octyldodecanol, oleic acid, oleyl alcohol, polyoxylglycerides, pyrrolidone, thymol, tricaprylin, triolein, myristic acid, medium chain triglycerides, linoleic acid, lauric acid, glycofurol, glyceryl monooleate, ethyl oleate, dimethyl sulfoxide, and dibutyl sebacate.

As used herein, an alkalizing agent is a component of the formulation that increases the pH of the mixture into which it is introduced. The formulations preferably include two alkalizing agents. The formulation preferably contains one or more alkalizing agents, such as ammonia solution, trolamine, tromethamine, sodium hydroxide, potassium hydroxide, diethanolamine, monoethanolamine, potassium citrate, sodium citrate, sodium bicarbonate, sodium borate, sodium carbonate, potassium bicarbonate, potassium carbonate, sodium acetate, sodium phosphate, and meglumine.

As used herein, an antimicrobial preservative is a component of the formulation that alone, or in conjunction with other components, helps to kill or inhibit the growth of microorganisms, such as bacteria, fungi, and/or yeasts. The formulation preferably contains one or more antimicrobial preservatives, such as methylparaben, ethylparaben, propylparaben, butylparaben, benzalkonium chloride, benzethonium chloride, cetylpyridinium chloride, benzoic acid, potassium benzoate, sodium benzoate, propionic acid, sodium propionate, potassium propionate, phenoxyethanol, phenylethyl alcohol, sorbic acid, sodium lactate, lactic acid, thymol, xylitol, imidurea, hexetidine, EDTA, cresol, chloroxylenol, chlorocresol, chlorobutanol, chlorhexidine, cetrimide, calcium lactate, calcium acetate, butylene glycol, bronopol, boric acid, and benzyl alcohol.

As used herein, an acidifying agent is a component of the formulation that decreases the pH of the mixture into which it is introduced. The formulation preferably contains one or more acidifyng agents such as hydrochloric acid, sulfuric acid, acetic acid, nitric acid, citric acid, propionic acid, adipic acid, lactic acid, phosphoric acid, tartaric acid, maleic acid, fumaric acid, calcium chloride, and ammonium chloride

As used herein, a gel-forming agent is a component of the formulation that, when dissolved/dispersed in a suitable solvent, forms a viscous gel. The formulation preferably contains one or more gel forming agents, such as carbomers (e.g., carbomer 940, or other grades of carbomer such as 934, 934P, 941, 1342) copolymer, homopolymer, interpolymer), polycarbophil, polyvinyl alcohol, povidone, hypromellose, sodium hyaluronate, hyaluronic acid, xanthan gum, pectin, methylcellulose, hydroxypropyl cellulose, hydroxyethylmethylcellulose, hydroxyethylcellulose, guar gum, dextrin, copovidone, ceratonia, carrageenan, alginic acid, carboxymethylcellulose sodium, carboxymethylcellulose calcium, ammonium alginate, sodium alginate, acacia, and potassium alginate.

As used herein, an oily internal phase (of emulsion) vehicle is a hydrophobic component of the formulation that alone, or in conjunction with other components, constitutes the internal (discontinuous) phase of an oil-in-aqueous emulsion. The formulation preferably contains one or more oily internal phase (of emulsion) vehicles, such as dimethicone, various grades of vegetable oil, mineral oil, isopropyl palmitate, octyldodecanol, oleyl alcohol, petrolatum, simethicone, tricaprylin, triolein, myristyl alcohol, medium chain triglycerides, glyceryl monooleate, ethyl oleate, dibutyl sebacate, and cyclomethicone.

As used herein, an ionic emulsifying agent is a component of the formulation that (1) contains at least one functional group that, at the pH of the mixture in which it is used, is substantially ionized, and (2) helps to form and/or stabilize the formulation of an emulsion. The formulation preferably contains one or more ionic emulsifying agents, such as stearic acid, oleic acid, palmitic acid, sodium lauryl sulfate, anionic emulsifying wax, myristic acid, linoleic acid, lecithin, lauric acid, docusate sodium, and aluminum monostearate.

As used herein, a non-ionic emulsifying agent is a component of the formulation that (1) contains no functional group that, at the pH of the mixture in which it is used, is substantially ionized, and (2) helps to form and/or stabilize the formulation of an emulsion. The formulation preferably contains one or more non-ionic emulsifying agents such as sorbitan monooleate, polyoxyethylene alkyl ethers, various polysorbate grades (20, 21, 40, 60, 61, 65, 80, 81, 85, and 120), polyoxyethylene castor oil derivatives, polyoxyethylene stearate, polyoxylglycerides, other sorbitan esters (laurate, palmitate, stearate, trioleate, sesquioleate, dioleate, sesquiisostearate, sesquistearate, triisostearate, tristearate, diisostearate, monoisostearate), nonionic emulsifying wax, myristyl alcohol, medium chain triglycerides, macrogol 15 hydroxystearate, glyceryl monooleate, cholesterol, cetyl alcohol, cetostearyl alcohol, mono and diglycerides, and triton X-100.

The inactive ingredients in the formulation may serve more than one function in the formulation.

The solvent (especially water) may also serve as a diluent, which means that it reduces the concentrations of the other components in the formulation. The solvent may also serve as a humectant, which is a component of the formulation which, when applied to the skin, causes moisture to be retained in the outer layers of the skin so as to increase its level of hydration. Some solvents may also serve as a stabilizer, which means that it helps prevent phase separation in an emulsion. Some solvents or oily internal phase vehicles may also serve as a lubricant, which means that, when applied to the skin, it imparts a slippery feeling. Some solvents may also serve as coating agent, which means that it helps the formulation to spread uniformly over the surface of the skin. Some solvents may also serve as an emollient, which means that it softens the skin.

The alkalizing agent/s may also serve as a solubilizing agent, which means that it increases the rate and/or extent to which another component in the formulation dissolves in solvent in which it is in contact with. The alkalizing agent may also serve as a buffering agent, which means that it causes a mixture to resist changes in pH when small amounts of acid or base are added.

The gel-forming agent and some solvents may also serve as a thickening agent, which means that, either alone or in conjunction with another component, it increases the viscosity of the mixture into which it is introduced.

Some oily internal phase vehicles may also serve as an antifoamant, which means that it helps the formulation to dissipate and/or prevent the formation of foams.

Some ionic emulsifying agents may also serve as a stiffening agent, which means that it increases the viscosity, especially of an emulsion.

In some embodiments, the present disclosure provides a method of administration comprising applying about 0.08 to about 1.0 grams of the topical pharmaceutical formulation described above to a mammal. In other embodiments of the method, about 0.1 to about 0.5 grams of the topical pharmaceutical formulation is applied to a mammal.

In some embodiments, the present disclosure provides a method of treating or preventing dermatoses associated with the administration of a 5-fluorouracil or a prodrug thereof in a mammal in need thereof by topically administering to the mammal in need thereof the formulation described above.

In some embodiments the mammal is a human, and the formulation is applied to a palm of the human. In some embodiments, the mammal is a human, and the formulation is applied to a sole of the human.

In some embodiments, the amount of formulation applied is about 0.333 grams or about 0.666 grams. In some embodiments, the amount of formulation applied is about 0.1 to about 0.7 grams, about 0.3 to about 0.4 grams, or about 0.5 to about 0.7 grams.

In some embodiments, the present disclosure provides a method of preventing Hand-Foot syndrome (HFS) associated with chemotherapy comprising administering the formulation to a palm and/or a sole of a mammal in need thereof, wherein said mammal is receiving systemic treatment with a 5-Fluoropyrimidine such as 5-fluorouracil, or a precursor or prodrug thereof, such as capecitabine. In some embodiments, the mammal is receiving systemic capecitabine. In some embodiments, the mammal is receiving 5-fluorouracil. In some embodiments the mammal is a human.

In some embodiments, the administration is performed twice daily during the period said mammal is receiving capecitabine therapy. In some embodiments, the administration first occurs about 5 about 30 minutes prior to capecitabine administration. In some embodiments, the administration first occurs about 15 minutes prior to capecitabine administration. Treatment is preferably started at least about 15 minutes before capecitabine administration, although it may begin earlier than this, and continues throughout capecitabine usage.

In some embodiments about one third of a gram of the formulation (which contains about 1 mg of uracil) is administered per two palms or per two soles. In some embodiments, about 0.8 mg to about 1.2 mg of uracil is administered per two palms, or per two soles.

The formulation is applied topically twice daily (BID) for 21 days per cycle to the palms of the hands and soles of the feet plus capecitabine 1000 mg/m2 orally (PO) BID on days 1 through 14 every 21 days.

The formulation is applied on a continuous schedule without interruption of treatment, while the mammal is receiving systemic treatment with a 5-Fluoropyrimidine such as 5-fluorouracil, or a precursor or prodrug thereof, such as capecitabine.

In some embodiments, the formulation of the invention is for use in treating or preventing dermatoses associated with the administration of a 5-fluorouracil or a prodrug thereof in a mammal in need thereof. In other embodiments, the formulation is for use in preventing Hand-Foot syndrome (HFS) associated with systemic chemotherapy, in a mammal in need thereof.

In some embodiments of these uses of the formulation, the mammal is a human, and the formulation is applied to a palm or sole of the human. In some embodiments, the amount of formulation applied is about 0.1 to about 0.5 grams. In other embodiments, the amount of formulation applied is about 0.3 to about 0.4 grams or about 0.5 to about 0.7 grams.

### Definitions

As used herein, “a” or “an” means one or more, unless specified otherwise. As used herein, when used in conjunction with the word “comprising,” the words “a” or “an” mean one or more than one, unless specified otherwise. As used herein, “another” or “a further” may mean at least a second or more.

When the term “about” is used in conjunction with a numerical value or range, it modifies that value or range by extending the boundaries above and below the numerical values set forth. In general, the term “about” is used herein to modify a numerical value above and below the stated value by a variance of 10 percent of the value, up or down (higher or lower), i.e., +10%, unless a different variance is indicated (e.g., +30%, +20%, +5%, +1%, +0.5% etc.).

“Percent” or “%” as used herein refers to weight (w/w) percentage unless otherwise specified.

The use of the term “or” in the claims is used to mean “and/or”, unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

Where features or aspects of the disclosure or claims are described in terms of Markush groups, those skilled in the art will recognize that the disclosure is also thereby described in terms of any individual member or subgroup of members of the Markush group.

In addition, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, and the like. As a non-limiting example, each range discussed herein can be readily broken down into a lower third, middle third and upper third, and the like. As will also be understood by one skilled in the art all language such as “up to,” “at least,” “greater than,” “less than,” include the number recited and refer to ranges which can be subsequently broken down into sub-ranges as discussed above. Finally, as will be understood by one skilled in the art, a range includes each individual member. For example, a group having 1-3 members refers to groups having 1, 2, or 3 members. Similarly, a group having 1-5 members refers to groups having 1, 2, 3, 4, or 5 members, and so forth.

As used herein, methyl methacrylate polymer refers to a synthetic polymer of methyl methacrylate (an organic methyl ester), called Poly(methacrylic acid methyl ester) (PMMA).

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

All references cited herein, including patents, patent applications, papers, textbooks and the like, and the references cited therein, to the extent that they are not already, are hereby incorporated herein by reference in their entirety.

### EXAMPLES

The disclosure is further illustrated by the following examples which are provided merely to be exemplary and do not limit the scope of the invention. Certain modifications and equivalents will be apparent to those skilled in the art and are intended to be included within the scope of the disclosure. The present disclosure provides, but is not limited to, the following examples.

### Example 1. Formulations

The four cream formulations described in Table 1, below, were made

TABLE 1
Uracil Cream Without Penetration Enhancer

| | | Placebo | Drug | Drug | Drug |
|---|---|---|---|---|---|
| | Composition No. | 1 | 2 | 3 | 4 |
| Phase | Ingredient | % (w/w) | % (w/w) | % (w/w) | % (w/w) |
| A | Uracil | x | 0.10 | 0.30 | 3.00 |
| A | Distilled Water-A | 10.10 | 10.00 | 10.10 | 10.00 |
| A | Ammonia Solution (29%) | 1.03 | 1.03 | 1.03 | 1.03 |
| B | Distilled Water-B | 10.87 | 10.87 | 10.87 | 10.87 |
| B | Methylparaben | 0.50 | 0.50 | 0.50 | 0.50 |
| B | Propylparaben | 0.05 | 0.05 | 0.05 | 0.05 |
| C | Distilled Water-C | 27.75 | 27.75 | 27.45 | 24.85 |
| C | Hydrochloric Acid (20% | q.s. | q.s. | q.s. | q.s. |
| | aqueous HCl) (q.s., was | | | | |
| | approximately 0.05% w/w) | | | | |
| C | Carbomer 940 | 1.11 | 1.11 | 1.11 | 1.11 |
| C | Trolamine (Triethanolamine) | 0.58 | 0.58 | 0.58 | 0.58 |
| D | Distilled Water-D | 10.00 | 10.00 | 10.00 | 10.00 |
| D | Polyethylene Glycol 400 | 14.85 | 14.85 | 14.85 | 14.85 |
| D | Glycerin | 13.43 | 13.43 | 13.43 | 13.43 |
| D | Propylene Glycol | 1.11 | 1.11 | 1.11 | 1.11 |
| E | Dimethicone | 3.30 | 3.30 | 3.30 | 3.30 |
| E | Stearic Acid | 2.84 | 2.84 | 2.84 | 2.84 |
| E | Polysorbate 80 | 1.45 | 1.45 | 1.45 | 1.45 |
| E | Sorbitan Monooleate | 1.03 | 1.03 | 1.03 | 1.03 |
| | Total | 100.00 | 100.00 | 100.00 | 100.00 |

Each of the formulations described in Table 1 above were prepared by the following procedure:

In an appropriately sized auxiliary vessel Phase A was prepared by combining the Phase A ingredients. The ingredients were mixed until Uracil was completely dissolved. The mixture was warmed slightly, below 50° C., to accelerate dissolution. This Phase A Auxiliary Vessel now contained completed Phase A. In another appropriately sized auxiliary vessel (Phase B Auxiliary Vessel), Phase B was prepared by combining the Phase B ingredients and heating to 50-60° C. The ingredients were mixed until the parabens were mostly dissolved. Phase A was transferred to the Phase B Auxiliary Vessel. The alkalinity of Phase A assisted with paraben dissolution. Phases A and B were mixed until all ingredients were dissolved, while the temperature was maintained at 50-60° C. The Phase B Auxiliary Vessel now contained the combined Phase A and Phase B ingredients. In another appropriately sized vessel (the Main Vessel), Phase C was prepared by adding the ingredients in the order listed. Carbomer 940 was sprinkled in with very rapid mixing. If gel bodies were present, mixture was lightly homogenized to achieve uniformity, and then Trolamine was added. After addition of Trolamine, Phase C became very thick and transparent. Phase C was heated to 50° C., and this temperature was maintained. In two separate appropriately sized auxiliary vessels, Phases D (in Auxiliary Vessel D) and E (in Auxiliary Vessel E) were prepared. Each of Phases D and E were heated to 80° C., and then Phase E was added to Phase D. Auxiliary Vessel D will now contain the combined phases E and D. The contents of Auxiliary Vessel D were cooled to 45-55° C. while mixing. The contents of Auxiliary Vessel D were added to Phase C in the Main Vessel. Main Vessel now contains combined Phases C, D, E.

While the contents of the Main Vessel were still warm (approximately 40-55° C.), Phases A and B from Auxiliary Vessel B were added to the Main Vessel, which now contains all Phases. The contents were mixed until uniform. A smooth, white, pearlescent cream was formed.

### Example 2. Preliminary Clinical Study of Uracil Formulation

To evaluate efficacy of topical application of uracil, Composition No. 2 in Table 1 above, a randomized, double-blind, placebo-controlled Phase 1-2 clinical study was conducted in 18 patients with metastatic breast cancer being treated with capecitabine. Nine of the patients were randomized to placebo (PTO, Composition 1 in Table 1). Patients were instructed to completely rub the formulation (Composition 2 (uracil), or Composition 1 (placebo)) into the palms of both hands and soles of both feet twice a day for each of two daily capecitabine treatments. Capecitabine was administered orally at its approved dose of 1250 mg/m² twice daily on Days 1-14 of each 21-day cycle. Treatment was continued for a maximum of 6 cycles unless tumor progression was documented, unacceptable toxicity emerged, or consent was withdrawn.

Adverse events (AEs), including Hand-Foot Syndrome (HFS), were assessed on days 1 and 15 of every dosing cycle and at study completion or discontinuation. Incidences of HFS were recorded according to the highest grade (NCI CCTAE scale) in each patient as a function of study arm.

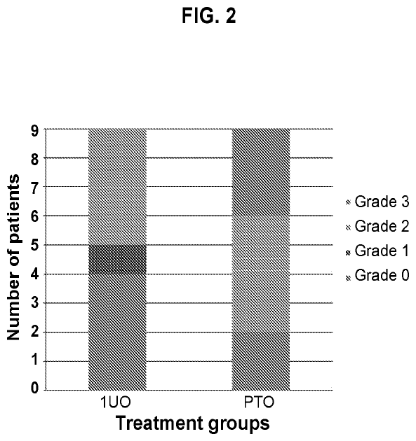

Results of the distribution of the incidences of HF S by the highest grade in each patient, as a function of study arm, are shown in FIG. 2.

TABLE 2

| | 1UO | Placebo | Total |
|---|---|---|---|
| | (n = 9) | (n = 9) | (n = 18) |
| Patients with ≥Grade 2 HFS, n (%) | 4 (44.4) | 7 (77.8) | 11 (61.1) |
| Capecitabine dose reduction or | 2 (22.2) | 6 (66.7) | 8 (44.4) |
| discontinuation following ≥Grade | | | |
| 2 HFS, n (%) | | | |
| Median time from randomization | 9.0 | 7.1 | |
| to ≥Grade 2 HFS, weeks | | | |
| 1UO: Treatment hazard ratio (95% | | | 0.33 (0.8, |
| CI) Log rank p-value (two-sided) | | | 1.28) 0.09 |

Table 2, above, summarizes the time from randomization to Grade≥2 HFS in each arm of the study. If a patient did not develop Grade≥2 HFS, the patient was censored as event-free at their last follow-up assessment. HFS of any grade was observed in five patients (55.5%) in the uracil cream (Composition 2) group and seven (77.8%) patients in the placebo group. HFS Grade≥2 was observed in four patients (44.4%) in the uracil cream group and seven patients (77.8%) in the PTO group. Two patients (22.2%) in the uracil cream group and six patients (66.7%) in the PTO group required dose reductions or discontinuation for Grade≥2 HFS. At the occurrence of Grade≥2 HFS, the XELODA prescriber information dictates an interruption or attenuation in capecitabine dosing as to avert the onset of severe Grade 3 HFS and more protracted treatment interruptions. Importantly, patients in the uracil cream arm had a significantly longer time to occurrence of Grade≥2 HFS (treatment group hazard ratio (HR)=0.33, p=0.09) when compared to patients in the PTO arm.

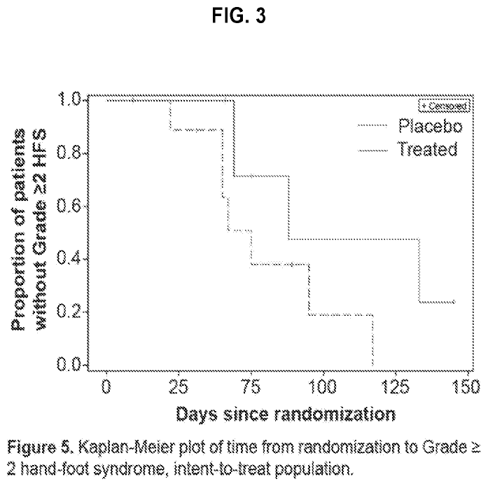

FIG. 3 shows a Kaplan-Meier plot of the proportion of patients without Grade≥2 HFS from randomization in each study arm. Although the small number of study patients precludes a precise quantification of the prophylactic effects of uracil cream on capecitabine-associated HFS, the study results indicate that uracil cream delays the onset of clinically-relevant grades of HFS. One patient in each arm experienced a single AE attributed to the topical cream and there were no serious adverse events (SAE) attributable to the investigational agent. A high proportion of patients in each study arm had AEs, most of which are well known to be caused by capecitabine and/or the underlying disease.

Thus, although the overall incidences of all grades of HFS were similar between the treatment and placebo arms, a higher proportion of patients treated with placebo experienced higher grades of HFS than patients who were treated with uracil cream, with higher grades of HFS occurred sooner after randomization in the PTO group. HFS was the cause of more capecitabine dose reductions and treatment interruptions in the PTO group when compared to the uracil cream group. Uracil cream was generally very well tolerated.

Uracil levels in patient plasma samples were investigated over time after application of one gram of uracil cream to the patients' palms and soles. The plasma concentration of uracil increased slowly from the baseline mean value of 19.20 ng/mL. Tₘₐₓ was reached at 2.5 hours with a mean concentration of 45.40 ng/mL. When comparing the Cₘₐₓ values of this study to the other reported oral administrations of uracil (500 mg/m²) (see van Staveren et al., Cancer Chemother Pharmacol 68: 1611-1617 (2011)), the oral route produced multiple orders of magnitude higher concentrations of uracil than found in this study (20 mg/L vs. 45.40 ng/mL). The baseline concentrations of uracil were almost identical to reported values reported in Bi et al., J Chromatogr B Biomed Sci Appl 738: 249-258 (2000), 19.20 vs. 19.06 ng/mL. Upon repeated applications of uracil cream for 56 days, the concentrations of uracil remained constant. This suggests that there was no accumulation of uracil in the studied patients. Capecitabine given at doses ranging from 800 to 1250 mg/m² produced Cₘₐₓ values for capecitabine and its metabolites in the mg/mL range (see Reigner et al., Clin Pharmacokinet 40: 85-104 (2001)). Any systemic perturbation in uracil concentrations resulting from 1UO application were negligible when compared to the systemic activity of capecitabine.

### Example 3. Protective Effect of Uracil against 5-FU in Cultured Human Keratinocytes

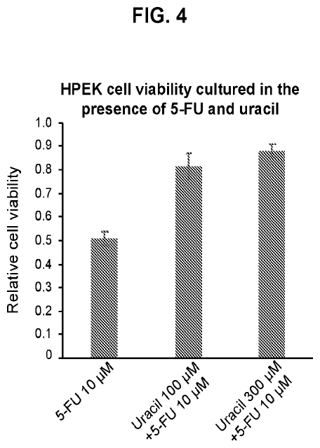

Primary human epidermal keratinocytes (HPEK cells) were cultured for 120 h under standard culture conditions in the presence of either 10 μM 5-FU, 10 μM 5-FU+100 μM uracil, or 10 μM 5-FU+300 μM uracil and cell viability determined after 120 h incubation. An increase in relative cell viability was observed with increasing uracil concentrations, as shown in FIG. 4.

### Example 4. Uracil Topical Cream Formulation and Manufacturing Information

A feasibility analysis found that the 1UO formulation (Composition 2 in Table 1), while effective, was not fully optimized. Therefore, several additional formulations were prepared. Formulation optimization efforts focused on developing a formula exhibiting (1) enhanced penetration of uracil into the skin with minimal permeation into the systemic circulation; and (2) improved aesthetics as measured by improved spreadability, faster drying time, and more favorable patient-friendly rheological properties. The formulation was engineered to allow for a facile, homogenous application with minimal rubbing and excellent coating and adherence to skin for proper and efficient absorption. The optimized creams are odorless and leave no noticeable residue or film on the hands and feet after application. 42 different formulations were considered and several lead formulations were tested using in vitro permeation test (IVPT) across human cadaver skin. Of these formulations, Composition 7 in Table 3, below, which contains dimethyl isosorbide (DMI) performed the best. This formula has been reproducibly prepared at laboratory scale (5 kg) with defined product specifications, analytical profile, and stability.

TABLE 3
With Penetration Enhancer

| Type | | Control | Drug | Drug | Drug | Control | Drug | Drug | Drug |
|---|---|---|---|---|---|---|---|---|---|
| No. | Composition No. | 5 | 6 | 7 | 8 | 9 | 10 | 11 | 12 |
| Phase | Ingredient | % (w/w) | % (w/w) | % (w/w) | % (w/w) | % (w/w) | % (w/w) | % (w/w) | % (w/w) |
| A | Uracil | X | 0.10 | 0.30 | 3.00 | x | 0.10 | 0.30 | 3.00 |
| A | Distilled Water-A | 10.00 | 10.00 | 10.00 | 10.00 | 10.00 | 10.00 | 10.00 | 10.00 |
| A | Gransolv DMI | 5.00 | 5.00 | 5.00 | 5.00 | 15.00 | 15.00 | 15.00 | 15.00 |
| A | Ammonia Solution | 1.03 | 1.03 | 1.03 | 1.03 | 1.03 | 1.03 | 1.03 | 1.03 |
| | (29%) | | | | | | | | |
| B | Methylparaben | 0.50 | 0.50 | 0.50 | 0.50 | 0.50 | 0.50 | 0.50 | 0.50 |
| B | Propylparaben | 0.05 | 0.05 | 0.05 | 0.05 | 0.05 | 0.05 | 0.05 | 0.05 |
| B | Polyethylene Glycol | 14.85 | 14.85 | 14.85 | 14.85 | 14.85 | 14.85 | 14.85 | 14.85 |
| | 400 | | | | | | | | |
| B | Glycerin | 13.43 | 13.43 | 13.43 | 13.43 | 13.43 | 13.43 | 13.43 | 13.43 |
| B | Propylene Glycol | 1.11 | 1.11 | 1.11 | 1.11 | 1.11 | 1.11 | 1.11 | 1.11 |
| C | Distilled Water-C | 43.72 | 43.62 | 43.42 | 40.72 | 33.72 | 33.62 | 33.42 | 30.72 |
| C | Hydrochloric Acid | q.s. | q.s. | q.s. | q.s. | q.s. | q.s. | q.s. | q.s. |
| | (20% aqueous) (q.s. | | | | | | | | |
| | is approximately | | | | | | | | |
| | 0.05%) | | | | | | | | |
| C | Carbomer 940 | 1.11 | 1.11 | 1.11 | 1.11 | 1.11 | 1.11 | 1.11 | 1.11 |
| C | Trolamine | 0.58 | 0.58 | 0.58 | 0.58 | 0.58 | 0.58 | 0.58 | 0.58 |
| | (Triethanolamine) | | | | | | | | |
| D | Dimethicone | 3.30 | 3.30 | 3.30 | 3.30 | 3.30 | 3.30 | 3.30 | 3.30 |
| D | Stearic Acid | 2.84 | 2.84 | 2.84 | 2.84 | 2.84 | 2.84 | 2.84 | 2.84 |
| D | Polysorbate 80 | 1.45 | 1.45 | 1.45 | 1.45 | 1.45 | 1.45 | 1.45 | 1.45 |
| D | Sorbitan | 1.03 | 1.03 | 1.03 | 1.03 | 1.03 | 1.03 | 1.03 | 1.03 |
| | Monooleate | | | | | | | | |
| | Total | 100.00 | 100.00 | 100.00 | 100.00 | 100.00 | 100.00 | 100.00 | 100.00 |

Each of the formulations described in Tables 1 and 2 above were prepared by the following procedure:

In an appropriately sized auxiliary vessel Phase A was prepared by combining the Phase A ingredients. The ingredients were mixed until Uracil was completely dissolved. The mixture was warmed slightly, below 50° C., to accelerate dissolution. This Phase A Auxiliary Vessel now contained completed Phase A. In another appropriately sized auxiliary vessel (Phase B Auxiliary Vessel), Phase B was prepared by combining the Phase B ingredients and heating to 50-60° C. The ingredients were mixed until the parabens were mostly dissolved. Phase A was transferred to the Phase B Auxiliary Vessel. The alkalinity of Phase A assisted with paraben dissolution. Phases A and B were mixed until all ingredients were dissolved, while the temperature was maintained at 50-60° C. The Phase B Auxiliary Vessel now contained the combined Phase A and Phase B ingredients. In another appropriately sized vessel (the Main Vessel), Phase C was prepared by adding the ingredients in the order listed. Carbomer 940 was sprinkled in with very rapid mixing. If gel bodies were present, mixture was lightly homogenized to achieve uniformity, and then Trolamine was added. After addition of Trolamine, Phase C became very thick and transparent. Phase C was heated to 50° C., and this temperature was maintained. In two separate appropriately sized auxiliary vessels, Phases D (in Auxiliary Vessel D) and E (in Auxiliary Vessel E) were prepared. Each of Phases D and E were heated to 80° C., and then Phase E was added to Phase D. Auxiliary Vessel D will now contain the combined phases E and D. The contents of Auxiliary Vessel D were cooled to 45-55° C. while mixing. The contents of Auxiliary Vessel D were added to Phase C in the Main Vessel. Main Vessel now contains combined Phases C, D, E.

While the contents of the Main Vessel were still warm (approximately 40-55° C.), Phases A and B from Auxiliary Vessel B were added to the Main Vessel, which now contains all Phases. The contents were mixed until uniform. A smooth, white, pearlescent cream was formed.

### Example 5. In Vitro Permeation Testing With Franz Cell Diffusion Apparatus

Dermal permeation testing across human cadaver skin is considered the best surrogate for in vivo human testing (Abd et al., Clinical Pharmacology 8: 163-176 (2016)). Dermatomed human cadaver skin from two donors (ages 60 and 57 years, 250 μM thickness) was used to compare two uracil formulations, Composition 7 (which includes the penetration enhancer dimethyl isosorbide) and Composition 2 (without a penetration enhancer). Each formulation was tested in triplicate on skin from each donor (using skin samples from the same cadaver for each set of triplicate), with untreated skin used to control for the amount of endogenous uracil. Prior to mounting on the Franz cell, skin was thawed to room temperature and cut into pieces of 28 mm diameter. 1.5 mg/cm² of each uracil formula (calculated based on the clinical dose) was applied to the skin surface and receiver compartment fluid was sampled at 0.5, 1, 2, 4, 6, 8, 10, and 12 hour time points and samples were subjected to mass spectrometry for quantification of the amount of uracil permeated across the skin. To measure uracil levels that had penetrated the skin, without permeating, after 12 h, the skin was biopsy punched and subjected to a washing protocol that had been previously validated to remove uracil on the skin surface. The weighed skin was cut into small pieces, vortexed for 16 h in 5 ml of aqueous ammonium hydroxide (pH 9) to extract the uracil, and centrifuged at 11000 rpm for 10 min. The extraction protocol had been previously validated (98+2% recovery) by extracting uracil that had been injected into the skin.

Of 42 formulations produced, the 6 in Table 4 below were selected for Franz testing. Similar permeation was observed, regardless of uracil % in topical cream. (range 80.1 to 143.6 ng/cm²). For % permeated, see table 4.

TABLE 4

| | 0.3 Uracil- | 0.3 Uracil- | 0.3 Uracil- | 0.1 Uracil- | 0.1 Uracil- | 0.1 Uracil- |
|---|---|---|---|---|---|---|
| Penetration | 15% DMI | 5% DMI | 0% DMI | 15% DMI | 5% DMI | 0% DMI |
| ng/mg | 0.47 | 1.40 | 0.43 | 0.22 | 0.19 | 0.19 |
| tissue/mg dose | | | | | | |

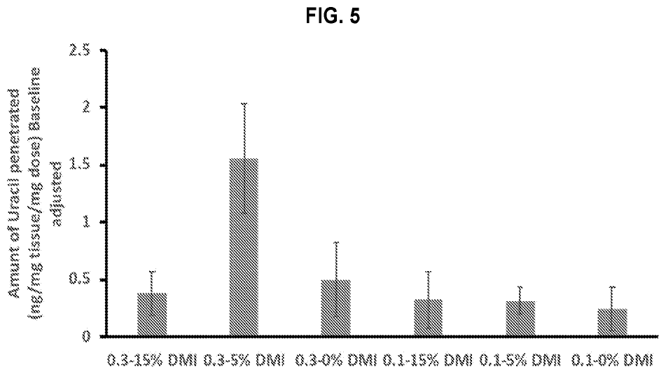

As shown in FIG. 5, the formulation with penetration enhancer (Composition 7) produced a 6.5-fold increase in uracil penetrated into the skin when compared to the uracil formula without penetration enhancer (Composition 2) (p=0.01) that was used in the Phase 1-2 clinical trial, as shown in FIG. 5. The cumulative amount of permeated uracil recovered from the receiver compartment at 12 h was nearly identical when comparing these two different uracil formulas. This suggests that like Composition 2, Composition 7 (with penetration enhancer) is unlikely to interfere with capecitabine efficacy by increasing systemic uracil levels in patients. As shown in FIG. 5, the formula containing 0.3% uracil and 5% DMI (Composition 7) outperformed all other formulations tested, even compared to the formulations that contained 15% DMI. Importantly, the total amount of permeated uracil recovered from the receiver compartment at 12 h was nearly identical when comparing all formulas. This indicates that like Composition 2 (without penetration enhancer), which does not cause systemic changes in uracil levels that would impact capecitabine efficacy, Composition 7, containing 5% DMI, would also be unlikely to impact the anti-cancer activity of capecitabine and its metabolites.

### Example 6. Large Batch Production of UTC

Further evaluation will be performed with 75 kg batches of uracil topical formulation (UTC). The scaled-up production will be tested by HPLC to ensure that there are no changes in the stability of uracil or preservatives as the batches are scaled. Accelerated and real-time stability studies will be performed at the ICH conditions 25° C./60% RH, 30° C./65% RH (back-up, no testing), and 40° C./75% RH. The test includes t=0 and 5 further time points over 3 months of formula alone and in the packaging selected for the clinical trial. The drug product will also be tested with forced degradation by heat, acid, base, oxidation and light (ambient and ICH conditions).

The required drug concentration in the impurity/related substance drug product extraction to enable detection of the related substances at the reporting threshold (determined based on product and dosing) will be assessed. The drug will be extracted from the formulation prepared at 100% of the target concentration and the percentage recovery from each formulation (n=6 each) will be calculated to confirm that the precision of the drug product extraction method is “fit for purpose.”

A preservative efficacy test (PET) will be carried out according to standard procedures (Pharmaceutical Microbiology Manual, 2014) with a single preservative system at the recommended preservative concentration and at a lower preservative concentration (e.g. 90%) of this level (to mimic preservative degradation over the shelf life). The preservative efficacy will be performed on cream with the drug at a single strength and the corresponding placebo (a total of 4 batches).

### Example 7. Additional Evaluation of Topical Uracil Creams

Data from a Phase 1-2 clinical study (described in Examples 2-3) of mBC patients receiving capecitabine showed attenuation in the development of HF S in patients treated with 1UO compared with those treated with PTO as well as an acceptable safety profile of 1UO relative to placebo, albeit in a small number of patients. A Phase 2 clinical study will be as a randomized, double-blind, placebo-controlled study, but with a sample size of 100 patients with histologically- or cytologically-confirmed mBC receiving capecitabine.

As pain is the core symptom of HFS, the primary objective of the study is to assess whether concurrent treatment with UTC (Composition 7) among women with mBC treated with capecitabine delays a meaningfully detrimental increase (measured as 100% increase in time to a ≥2 point increase in 24-hour worst pain in the hands or feet for UTC relative to placebo) in patient-reported pain in the hands or feet expected to be associated with the development of HFS. The trial utilizes a Patient Reported Outcome (PRO) assessment of pain, based on item 3 of the Brief Pain Inventory (BPI) as the primary efficacy endpoint. This trial will enable the PRO outcome of pain intensity associated with HFS to be validated and serve as the primary end-point in a registrational clinical study. Time to the emergence of ≥Grade 2 HFS will be evaluated as a secondary endpoint. The safety and PK profiles of UTC as well as capecitabine and its metabolites will be investigated in this larger population of patients. Patients will receive capecitabine at 1000 mg/m² rather than the registered dose of 1250 mg/m², but on the same regimen (BID on D1 to 14 of a 21 day cycle). Clinical studies have shown a capecitabine dose of 1000 mg/m² to improve tolerability compared to a dose of 1250 mg/m² without diminished efficacy (Leonard et al., Clin Breast Cancer 11: 349-356 (2011); Zielinski et al., Ann Oncol 21: 2145-2152 (2010)). In the US as well as Canada and elsewhere the approved monotherapy starting dose for capecitabine of 1250 mg/m² is rarely used due to its association with an unacceptably high rate of HFS, diarrhea, mucositis, and other toxicities.

In addition to patient reported outcome of pain, patients will also be evaluated for HFS via physical examination and digital photography, with assessment of HFS severity using NCI CTCAE criteria shown below in Table 5. HFS will be evaluated on days 1 and 15 of each treatment cycle and at the end of the treatment visit. In the Phase 1-2 study, although UTC appeared to delay the development of clinically relevant HFS, UTC-treated patients appeared to develop various other dose-related capecitabine-associated AEs. Based on the experience with capecitabine in the U.S. and trends towards use of capecitabine doses below<1250 mg/m² BID on D1 to 14 of each 21-day cycle, it is likely that the overall therapeutic benefit of UTC in preventing HFS would be greater in patients receiving a lower dose of capecitabine (1000 mg/m² twice daily on D1 to 14 of each 21-day cycle), a dose regimen that is still associated with cumulative HFS, but with lower incidences of other capecitabine-related AEs.

TABLE 5
NCI CTCAE Grading Scale for HFS

| Grade | Classification |
|---|---|
| 1 | Minimal skin changes or dermatitis (e.g., erythema, |
| | edema, or hyperkeratosis) without pain. |
| 2 | Skin changes (e.g., peeling, blisters, bleeding, edema, |
| | or hyperkeratosis) with pain; limiting instrumental ADL. |
| 3 | Severe skin changes (e.g., peeling, blisters, bleeding, |
| | edema, or hyperkeratosis) with pain; limiting self care |
| | ADL. |

To assess the plasma PK of uracil, capecitabine, and capecitabine metabolites (5′-DFCR, 5′-DFUR and 5-FU), blood samples will be collected from the first 24 patients enrolled in each of the treatment groups: (a) C1D1: before UTC/PTC and capecitabine administration and 0.5, 1, 2, 4, 6, and 8 h after capecitabine administration; (b) C1D14: before UTC/PTC and capecitabine administration and 0.5, 1, 2, 4, 6, and 8 h after capecitabine administration. Additional blood samples to assess plasma concentrations of uracil, capecitabine, and capecitabine metabolites will be collected from all patients remaining in the study on D1C2 and subsequent even cycles, before and 2 h after UTC/PTC and capecitabine administration. Parameters including but not limited to Cₘₐₓ, Tₘₐₓ, AUC, Cl/F, V_{z}/F, t_{1/2}AUC, T_{1/2,} Cl, and V_{d} will be determined.

What is claimed is:

1. A topical pharmaceutical formulation comprising:
about 0.05 to about 0.8% w/w Uracil,
about 2 to about 8% w/w of a penetration enhancer,
about 0.01 to about 4% w/w of an alkalizing agent,
about 0 to about 5% w/w of an antimicrobial preservative,
about 10 to about 40% w/w of a solvent selected from the group consisting of polyethylene glycol 400, glycerin, propylene glycol, and mixtures thereof;
about 0.01 to about 3% w/w of an acidifying agent,
about 0.1 to about 3% w/w of a gel forming agent selected from the group consisting of a carbomer, polycarbophil, polyvinyl alcohol, povidone, hypromellose, sodium hyaluronate, hyaluronic acid, xanthan gum, pectin, methylcellulose, hydroxypropyl cellulose, hydroxyethylmethylcellulose, hydroxyethylcellulose, guar gum, dextrin, copovidone, ceratonia, carrageenan, alginic acid, carboxymethylcellulose sodium, carboxymethylcellulose calcium, ammonium alginate, sodium alginate, acacia, and potassium alginate, and mixtures thereof,
about 1 to about 5% w/w of an oily internal phase vehicle,
about 0 to about 5% w/w of an ionic emulsifying agent,
about 0 to about 7% w/w of a nonionic emulsifying agent, and
about 40 to about 70% w/w water.

2. The topical pharmaceutical formulation of claim 1, wherein the permeation of uracil from said formulation is less than about 150.6 ng/cm², as measured using IVTP.

3. The topical pharmaceutical formulation of claim 1, wherein the penetration enhancer is selected from the group consisting of dimethyl isosorbide, isopropyl myristate, isopropyl palmitate, octyldodecanol, oleic acid, oleyl alcohol, polyoxylglycerides, pyrrolidone, thymol, tricaprylin, triolein, myristic acid, medium chain triglycerides, linoleic acid, lauric acid, glycofurol, glyceryl monooleate, ethyl oleate, dimethyl sulfoxide, dibutyl sebacate, and mixtures thereof.

4. The topical pharmaceutical formulation of claim 1, wherein the alkalizing agent is selected from the group consisting of ammonia solution, trolamine, tromethamine, sodium hydroxide, potassium hydroxide, diethanolamine, monoethanolamine, potassium citrate, sodium citrate, sodium bicarbonate, sodium borate, sodium carbonate, potassium bicarbonate, potassium carbonate, sodium acetate, sodium phosphate, meglumine, and mixtures thereof.

5. The topical pharmaceutical formulation of claim 1, wherein the antimicrobial preservative is selected from the group consisting of methylparaben, ethylparaben, propylparaben, butylparaben, benzalkonium chloride, benzethonium chloride, cetylpyridinium chloride, benzoic acid, potassium benzoate, sodium benzoate, propionic acid, sodium propionate, potassium propionate, phenoxyethanol, phenylethyl alcohol, sorbic acid, sodium lactate, lactic acid, thymol, xylitol, imidurea, hexetidine, EDTA, cresol, chloroxylenol, chlorocresol, chlorobutanol, chlorhexidine, cetrimide, calcium lactate, calcium acetate, butylene glycol, bronopol, boric acid, benzyl alcohol, and mixtures thereof.

6. The topical pharmaceutical formulation of claim 1, wherein the acidifying agent is selected from the group consisting of hydrochloric acid, sulfuric acid, acetic acid, nitric acid, citric acid, propionic acid, adipic acid, lactic acid, phosphoric acid, tartaric acid, maleic acid, fumaric acid, calcium chloride, ammonium chloride, and mixtures thereof.

7. The topical pharmaceutical formulation of claim 1, wherein the oily internal phase vehicle is selected from the group consisting of dimethicone, various grades of vegetable oil, mineral oil, isopropyl palmitate, octyldodecanol, oleyl alcohol, petrolatum, simethicone, tricaprylin, triolein, myristyl alcohol, medium chain triglycerides, glyceryl monooleate, ethyl oleate, dibutyl sebacate, cyclomethicone, and mixtures thereof.

8. The topical pharmaceutical formulation of claim 1, wherein the ionic emulsifying agent is selected from the group consisting of stearic acid, oleic acid, palmitic acid, sodium lauryl sulfate, anionic emulsifying wax, myristic acid, linoleic acid, lecithin, lauric acid, docusate sodium, aluminum monostearate, and mixtures thereof.

9. The topical pharmaceutical formulation of claim 1, wherein the nonionic emulsifying agent is selected from the group consisting of: sorbitan monooleate; polyoxyethylene alkyl ethers; a polysorbate; a polyoxyethylene castor oil derivative; polyoxyethylene stearate; a polyoxylglyceride; a laurate, palmitate, stearate, trioleate, sesquioleate, dioleate, sesquiisostearate, sesquistearate, triisostearate, tristearate, diisostearate, or monoisostearate sorbitan ester; a nonionic emulsifying wax; myristyl alcohol; a medium chain triglyceride; macrogol 15 hydroxystearate; glyceryl monooleate; cholesterol; cetyl alcohol; cetostearyl alcohol; a monoglyceride; a diglyceride; triton X-100; and mixtures thereof.

10. The topical pharmaceutical formulation of claim 1, wherein the penetration enhancer is dimethyl isosorbide, and the ratio of the w/w concentrations of the uracil to dimethyl isosorbide is about 0.3 to 5.

11. The topical pharmaceutical formulation of claim 1, wherein the penetration enhancer is dimethyl isosorbide, and the concentration of uracil is about 0.3% w/w, and the concentration of dimethyl isosorbide is about 5.0% w/w.

12. The topical pharmaceutical formulation of claim 1, wherein the formulation is an emulsion, and wherein the viscosity of the formulation is about 100,000 to about 400,000 cps.

13. The topical pharmaceutical formulation of claim 1, wherein the formulation is an emulsion, and wherein the viscosity of the formulation is about 250,000 to about 320,000 cps.

14. A topical pharmaceutical formulation comprising:
about 0.05 to about 0.6% w/w Uracil,
about 3.0 to about 10% w/w Dimethyl isosorbide,
about 0.1 to about 2% w/w Ammonia Solution (about 29%),
about 0 to about 2% w/w Methylparaben,
about 0 to about 2% w/w Propylparaben,
about 10 to about 20% w/w Polyethylene Glycol,
about 10 to about 20% w/w Glycerin,
about 0 to about 3% w/w Propylene Glycol,
about 0 to about 3% w/w Hydrochloric Acid (about 20%),
about 0.1 to about 5% w/w Carbomer,
about 0.1 to about 2% w/w Trolamine,
about 1 to about 5% w/w Dimethicone,
about 1 to about 7% w/w a nonionic emulsifying agent,
about 0 to about 5% w/w an ionic emulsifying agent, and
about 40 to about 80% w/w Water.

15. The topical pharmaceutical formulation of claim 14, wherein the concentration of uracil is about 0.3% w/w, and the concentration of dimethyl isosorbide is about 5.0% w/w.

16. The topical pharmaceutical formulation of claim 14, wherein the ratio of the w/w concentrations of the uracil to dimethyl isosorbide is about 0.3 to 5.

17. The topical pharmaceutical formulation of claim 14, wherein the formulation is an emulsion, and wherein the viscosity of the formulation is about 100,000 to about 400,000 cps.

18. The topical pharmaceutical formulation of claim 14, wherein the formulation is an emulsion, and wherein the viscosity of the formulation is about 250,000 to about 320,000 cps.

19. A topical pharmaceutical formulation comprising:
about 0.05 to about 0.6% w/w Uracil,
about 3.0 to about 10% w/w Dimethyl isosorbide,
about 0.1 to about 4% w/w an alkalizing agent,
about 0.1 to about 2% w/w Methylparaben,
about 0.01 to about 1% w/w Propylparaben,
about 10 to about 40% w/w of a solvent selected from the group consisting of Polyethylene Glycol 400, Glycerin, Propylene Glycol, and mixtures thereof;
about 0.01 to about 3% w/w an acidifying agent,
about 0.1 to about 3% w/w Carbomer 940,
about 1 to about 5% w/w Dimethicone,
about 1 to about 5% w/w Stearic Acid,
about 0.5 to about 4% w/w Polysorbate 80,
about 0.1 to about 3% w/w Sorbitan Monooleate, and
about 40 to about 60% w/w Water.

20. A topical pharmaceutical formulation comprising:
about 0.05 to about 0.5% w/w Uracil,
about 3.0 to about 8% w/w Dimethyl isosorbide,
about 0.1 to about 2% w/w Ammonia Solution (about 29%),
about 0.1 to about 2% w/w Methylparaben,
about 0.01 to about 1% w/w Propylparaben,
about 10 to about 20% w/w Polyethylene Glycol 400,
about 10 to about 20% w/w Glycerin,
about 0.1 to about 3% w/w Propylene Glycol,
about 0.01 to about 3% w/w Hydrochloric Acid (about 20%),
about 0.1 to about 3% w/w Carbomer 940,
about 0.1 to about 2% w/w Trolamine,
about 1 to about 5% w/w Dimethicone,
about 1 to about 5% w/w Stearic Acid,
about 0.5 to about 4% w/w Polysorbate 80,
about 0.1 to about 3% w/w Sorbitan Monooleate, and
about 40 to about 60% w/w Water.

21. The topical pharmaceutical formulation of claim 20, wherein the concentration of uracil is about 0.3% w/w, and the concentration of dimethyl isosorbide is about 5.0% w/w.

22. A topical pharmaceutical formulation comprising:
about 0.3% w/w Uracil,
about 5.0% w/w Dimethyl isosorbide,
about 0.9 to about 1.1 w/w Ammonia Solution (about 29%),
about 0.4 to about 0.6% w/w Methylparaben,
about 0.04 to about 0.06% w/w Propylparaben,
about 14 to about 16% w/w Polyethylene Glycol 400,
about 13 to about 15% w/w Glycerin,
about 1 to about 2% w/w Propylene Glycol,
about 0.01 to about 0.1% w/w Hydrochloric Acid (about 20%),
about 1 to about 2% w/w Carbomer 940,
about 0.4 to about 0.6% w/w Trolamine,
about 3 to about 4% w/w Dimethicone,
about 2 to about 3% w/w Stearic Acid,
about 1 to about 2% w/w Polysorbate 80,
about 0.9 to about 2% w/w Sorbitan Monooleate, and
about 50 to about 60% w/w Water.

23. The topical pharmaceutical formulation of any of claims 1-14 and 19, wherein said formulation does not comprise a methyl methacrylate polymer.

24. A method of administration comprising applying about 0.08 to about 1.0 grams of the topical pharmaceutical formulation of any one of claims 1-23 to a mammal.

25. A method of administration comprising applying about 0.1 to about 0.5 grams of the topical pharmaceutical formulation of any one of claims 1-23 to a mammal.

26. The method of claim 25, wherein the mammal is a human, and wherein the formulation is applied to a palm of the human.

27. The method of claim 25, wherein the mammal is a human, and wherein the formulation is applied to a sole of the human.

28. The method of claim 25, wherein the amount of formulation applied is about 0.3 to about 0.4 grams or about 0.5 to about 0.7 grams.

29. A method of treating or preventing dermatoses associated with the administration of a 5-fluorouracil or a prodrug thereof in a mammal in need thereof by topically administering to the mammal in need thereof the formulation of any one of claims 1-23.

30. The method of claim 29, wherein the mammal is a human, and wherein the formulation is applied to a palm of the human.

31. The method of claim 29, wherein the mammal is a human, and wherein the formulation is applied to a sole of the human.

32. The method of claim 29, wherein the amount of formulation applied is about 0.3 to about 0.4 grams or about 0.5 to about 0.7 grams.

33. A method of preventing Hand-Foot syndrome (HFS) associated with systemic chemotherapy comprising administering the formulation of any one of claims 1-23 to a palm and/or a sole of a human in need thereof, wherein said human is receiving systemic chemotherapy that can cause HFS.

34. The method of claim 33, wherein said human is receiving systemic treatment with capecitabine.

35. The method of claim 34, wherein said administration is performed twice daily during the period said human is receiving capecitabine therapy.

36. The method of claim 35, wherein said administration first occurs about 5 to about 30 minutes prior to capecitabine administration.

37. The method of claim 33, wherein said human is receiving systemic treatment with 5-fluorouracil.

38. A formulation according to any one of claims 1-23, for use in treating or preventing dermatoses associated with the administration of a 5-fluorouracil or a prodrug thereof in a mammal in need thereof.

39. The formulation of claim 38, wherein the mammal is a human, and wherein the formulation is applied to a palm or sole of the human.

40. The formulation of claim 39, wherein the amount of formulation applied is about 0.1 to about 0.5 grams.

41. The formulation of claim 39, wherein the amount of formulation applied is about 0.3 to about 0.4 grams or about 0.5 to about 0.7 grams.

42. A formulation according to any one of claims 1-23, for use in preventing Hand-Foot syndrome (HFS) associated with systemic chemotherapy, in a mammal in need thereof.

43. The formulation of claim 42, wherein the mammal is a human, and wherein the formulation is applied to a palm or sole of the human.

44. The formulation of claim 43, wherein the amount of formulation applied is about 0.1 to about 0.5 grams.

45. The formulation of claim 43, wherein the amount of formulation applied is about 0.3 to about 0.4 grams or about 0.5 to about 0.7 grams.
